(19) United States
(12) Patent Application Publication
(10) Pub. No.: US 2007/0173909 A1
(43) Pub. Date: Jul. 26, 2007

(54) Spark gap in an implantable medical device

(75) Inventors: D. Michael Inman, Seabrook, TX (US); Bryan P. Byerman, League City, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/337,987

(22) Filed: Jan. 24, 2006

(51) Int. Cl. A61N 1/00

(52) U.S. Cl. 607/63

(57) ABSTRACT

An implantable medical device comprises an enclosure containing a gas and a plurality of conductors that couple to tissue. At least two of the conductors define a spark gap formed therebetween and exposed to the gas.

### BACKGROUND

Implantable medical devices are typically limited to relatively low working voltages. During handling in manufacturing and surgical implantation, however, such devices may be susceptible to electrostatic discharge (ESD) of, for example, 1000 volts or more. If such ESD is allowed to reach sensitive internal components, the operation of the medical device could be impaired. Although rarely a problem, ESD should not be ignored, and more effective solutions to the problem of ESD are needed.

### BRIEF SUMMARY

In accordance with at least one embodiment of the invention, an implantable medical device (IMD) comprises an enclosure containing a gas and a plurality of conductors that couple to tissue. At least two of the conductors define a spark gap formed therebetween and are exposed to the gas. Excessive levels of ESD will discharge through one or more of the spark gaps without damaging circuitry (e.g., control electronics) included within the IMD.

In accordance with another embodiment, an implantable medical device comprises a can, a circuit board contained within the can, control logic provided on the circuit board, a plurality of connection points, a plurality of conductive elements, and a gap formed between two conductive elements. Each connection point is adapted to couple to one of a lead and the can. Each conductive element electrically couples to a connection point. The gap is formed between the two conductive elements on an exposed surface of the circuit board. The gap is configured so as to encourage an electrostatic discharge arc from one of the two conductive elements to the other of the two conductive elements when a voltage on one of the conductive elements exceeds a safety threshold for the medical device.

Another embodiment is directed to a circuit board adapted to be housed within an enclosure of an implantable medical device. The circuit board preferably comprises control logic provided on the circuit board, a plurality of connection points, and a spark gap formed between two conductive elements on an exposed surface of the circuit board. Each connection point is adapted to couple to one of a lead and an enclosure. The spark gap is configured so as to cause an electrostatic discharge arc from one conductive element to another when a voltage on one of the conductive elements exceeds a safety threshold.

### BRIEF DESCRIPTION OF THE DRAWINGS

For a detailed description of exemplary embodiments of the invention, reference will now be made to the accompanying drawings in which:

FIG. 1 depicts, in schematic form, an implantable medical device, in accordance with a preferred embodiment of the invention, implanted within a patient and programmable by an external programming system;

FIG. 2 shows an embodiment of the invention in which one or more spark gaps are provided on a circuit board inside an enclosure of an implantable medical device to ameliorate the effects of ESD;

FIG. 3 shows an exemplary embodiment of a configuration for a spark gap;

FIG. 4 shows another embodiment of a configuration for a spark gap;

FIG. 5 is a cross-sectional view of a circuit in accordance with an embodiment of the invention;

FIG. 6 is a partial cross-sectional view showing a header mated to the enclosure of the implantable medical device;

FIGS. 7 and 8 are perspective and end views, respectively, showing a feedthrough component, at least part of which resides within the header, in which one or more spark gaps are provided;

FIG. 9 illustrates an embodiment in which a spark gap is provided in a cavity formed within a circuit board; and

FIG. 10 is a schematic view showing an embodiment in which diodes are provided in parallel with the spark gaps.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and is not intended to imply that the scope of the disclosure, including the claims, is limited to that embodiment. Any numerical dimensions and/or material specifications provided herein are merely exemplary and do not limit the scope of this disclosure or the claims that follow, unless otherwise stated.

In the disclosure and claims that follow, the terms “couple” and “coupled” include direct and indirect electrical connections. Thus, component A couples to component B, regardless of whether component A is connected directly to component B, or is connected to component B via one or more intermediate components or structures.

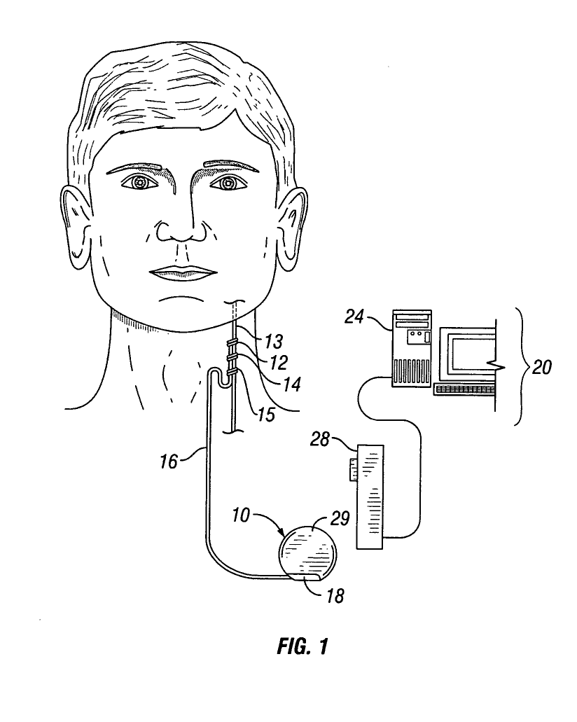

FIG. 1 illustrates an implantable medical device (“IMD”) 10 implanted in a patient. The IMD 10 may be representative of any of a variety of medical devices. At least one preferred embodiment of the IMD 10 comprises a neurostimulator for applying an electrical signal to a neural structure in a patient, particularly a cranial nerve such as a vagus nerve 13. Although the device 10 is described below in terms of vagus nerve stimulation (“VNS”), the disclosure and claims that follow, unless otherwise stated, are not limited to VNS, and may be applied to the delivery of an electrical signal to modulate the electrical activity of other cranial nerves such as the trigeminal and/or glossopharyngeal nerves, or to other neural tissue such as one or more brain structures of the patient, spinal nerves, and other spinal structures. Further still, the IMD 10 can be used to stimulate tissue other than nerves or neural tissue. An example of such other tissue comprises cardiac tissue.

Referring still to FIG. 1, a lead assembly comprising one or more leads 16 is coupled to the IMD 10 and includes one or more electrodes, such as electrodes 12 and 14. Each lead 16 has a proximal end that connects to a header 18 of the IMD 10 and a distal end on which one or more electrodes are provided. The outer enclosure (or “can”) 29 of the IMD 10 may be electrically conductive and thus may also function as an electrode in some embodiments. The electrodes 12, 14 and can 29 couple to the patient's tissue. The header 18 mates with the can 29. The header 18 contains one or more connectors to which the lead(s) 16 connect. Through conductive structures housed in the header 18, the leads electrically couple to circuitry inside the can. In at least one embodiment, the internal circuitry is implemented in the form of electrical components mounted on a printed circuit board. The electrodes, such as electrodes 12, 14 and can 29, can be used to stimulate and/or sense the electrical activity of the associated tissue (e.g., the vagus nerve 13). An example of an electrode suitable for coupling to a vagus nerve to provide VNS therapy to a patient is disclosed in U.S. Pat. No. 4,979,511, incorporated herein by reference. Strain relief tether 15 comprises an attachment mechanism that attaches the lead assembly 16 to the vagus nerve to provide strain relief and is described in U.S. Pat. No. 4,979,511, incorporated herein by reference.

FIG. 1 also illustrates an external device implemented as a programming system 20 for the IMD 10. The programming system 20 comprises a processing unit coupled to a wand 28. The processing unit 24 may comprise a personal computer, personal digital assistant (PDA) device, or other suitable computing device consistent with the description contained herein. Methods and apparatus for communication between the IMD 10 and an external programming system 20 are known in the art. Representative techniques for such communication are disclosed in U.S. Pat. No. 5,304,206, and U.S. Pat. No. 5,235,980, both incorporated herein by reference. The IMD 10 includes a transceiver (e.g., a coil) that permits signals to be communicated wirelessly and noninvasively between the external wand 28 and the implanted IMD 10. Via the wand 28, the programming system 20 generally monitors the performance of the IMD and downloads new programming information into the device to alter its operation as desired.

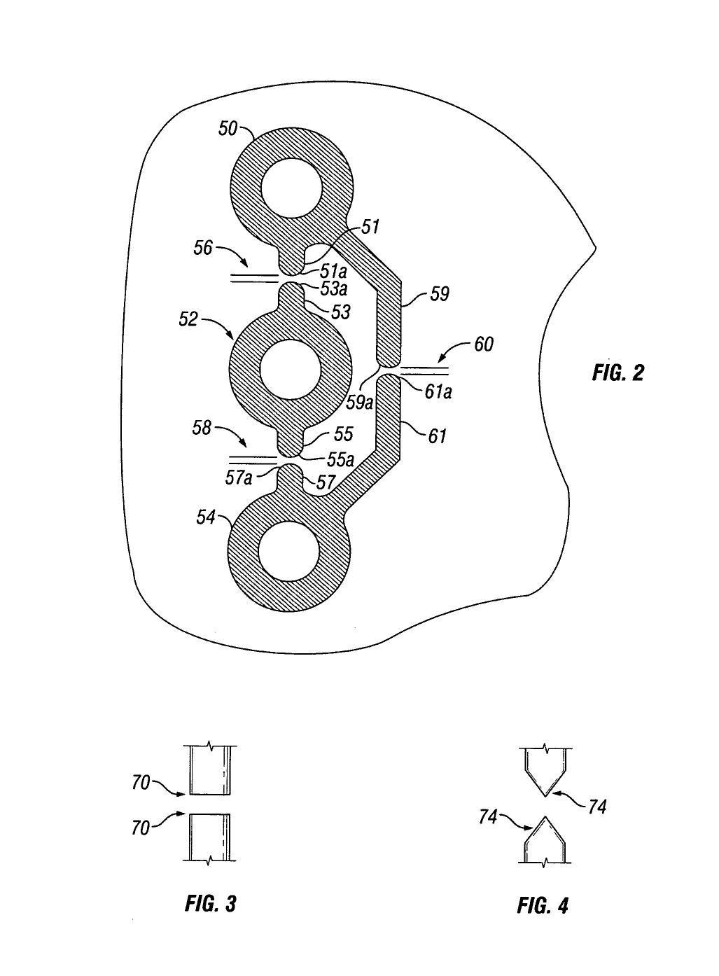

FIG. 2 shows a view of at least a portion of a circuit board 40 contained within the can 29 of the IMD 10. In accordance with at least some embodiments, three electrodes can be coupled to the IMD 10, although the number of electrodes is irrelevant to the scope of this disclosure. The three electrodes include, for example, the can 29 and two electrodes provided on leads 16. The three electrodes electrically couple directly or indirectly to the circuit board 40 at conductive pads 50, 52, and 54. Conductive pads 50-54 function as connection points for the leads or conductors coupled to the leads. The conductive pads thus comprise conductors that electrically couple to the patient's tissue(s) by way of the electrodes 12, 14, and 29. The conductive pads are formed from, for example, copper or other suitable conductive material and are provided on an exposed surface of the circuit board in accordance with known circuit board fabrication techniques. Conductive traces (not specifically shown) couple the conductive pads 50-54, and thus the electrodes 12, 14, 29, to communication circuitry, control logic, combinations thereof, and/or other circuitry that may be provided on the circuit board 40.

Referring still to FIG. 2, one or more conductive traces from each conductive pad 50-54 extend away from the associated conductive pad and towards a conductive trace associated with another conductive pad. In the exemplary embodiment of FIG. 2, conductive traces 51 and 59 extend away from conductive pad 50. Conductive traces 53 and 55 extend away from conductive pad 52, while conductive traces 57 and 61 extend away from conductive pad 54. Each such conductive trace 51, 53, 55, 57, 59, and 61 includes an end 51a, 53a, 55a, 57a, 59a, and 61a, respectively. Each conductive trace from a conductive pad extends toward, but does not electrically couple to, a trace from another conductive pad, thereby forming a gap between the ends of the traces. As shown in FIG. 2, gap 56 is formed between ends 51a and 53a of traces 51 and 53. Gap 58 is formed between ends 55a and 57a. Gap 60 is formed between ends 59a and 61a. Although three gaps are illustrated in the embodiment of FIG. 2, broadly, at least two of the conductive pads define at least one gap formed therebetween. Thus, at least one but, if desired, more than one gap is provided between pairs of conductive pads.

Each gap 56, 58, and 60 creates a “spark” gap to create an environment in which a sufficiently high electrical energy (e.g., ESD) imposed on an electrode will arc across the gap to another electrode instead of through the IMD's electronics, which could otherwise be damaged by ESD. In the embodiment of FIG. 2, because a spark gap is provided between each pair of electrodes, ESD on any one electrode can arc to any one or more other electrodes. For example, ESD from an electrode connected to conductive pad 50 can arc to conductive pad 52 via spark gap 56 and/or to conductive pad 54 via spark gap 60.

The IMD can 29 may be constructed from titanium and preferably is welded shut in an inert gas (e.g., argon) environment to avoid nitrogen weld embrittlement. The gas that remains sealed within the can 29 provides a gaseous environment to facilitate ESD to arc across a spark gap. An inert gas, such as argon, has a lower dielectric strength than nitrogen or room air, which means that in an argon environment, an electrical spark will arc a longer distance at a lower voltage than in a nitrogen or room air environment. Although an inert gas is preferred, other gasses (e.g., air) can be used as well.

In FIG. 2, the ends of the conductive traces that define the spark gaps 56, 58, and 60 are curved (i.e., not planar). FIG. 3 illustrates another embodiment in which the conductive trace ends 70 are square and extend substantially parallel to one another. In FIG. 4, the conductive trace ends 74 are formed to have an apex and may therefore be described as pointed. The shape of the conductive trace ends can be as shown in FIGS. 2-4 or in accordance with other shapes and configurations as desired, and may comprise combinations of such shapes and configuration. For example, one trace end defining a spark gap may be curved, while the corresponding other end is square.

The size of each spark gap (i.e., the distance between the closest portions of the adjacent ends of the traces that define each spark gap), the shape of the ends of the traces that define each spark gap, and the type and pressure of gas chosen to be sealed within the can determine the energy level at which ESD will arc across a spark gap. In at least one embodiment, the size of each spark gap is within the range of approximately 0.002 inches to 0.004 inches, and is preferably approximately 0.003 inches in some embodiments, in an argon gas environment at a pressure of 760 torr. As such, a voltage of approximately 220 volts or greater across a pair of, conductive pads 50-54 will arc across the spark gap provided between the pair of conductive pads. The size of the spark gaps and the selected conductive pad material, gas and pressure can be varied as desired. In one embodiment, copper is used for the conductive pads.

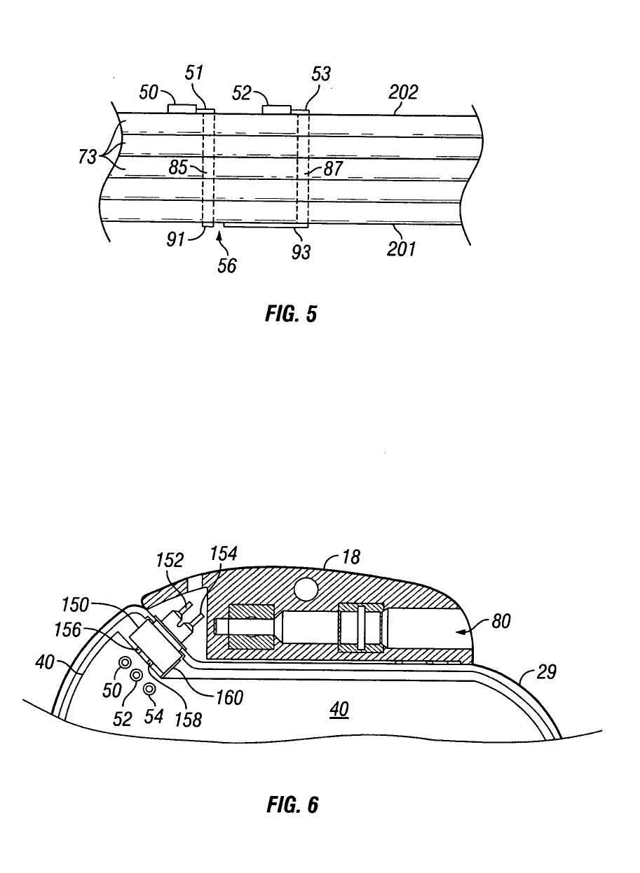

The traces shown in FIG. 2 as defining the spark gaps 56, 58, and 60 may comprise traces on a surface of circuit board 40. In some embodiments, the circuit board 40 may comprise multiple layers such as a top layer, a bottom layer, and one or more intermediate layers. The top and bottom layers comprise exposed surfaces of the circuit board 40. FIG. 5, for example, shows a cross-sectional view of circuit board 40. As shown, the board comprises multiple layers 73, a top exposed layer 202 and a bottom exposed layer 201. Conductive pads 50 and 52 and associated conductive traces 51 and 53 (discussed above) are also shown. Electrical connections are made from each conductive trace 51 and 53 through the various layers 73 (by way of “vias”) of the board 40 to corresponding conductive traces 91 and 93 that create a spark gap 56 therebetween. Accordingly, a spark gap formed between a pair of conductive pads may be provided on a surface of the circuit board opposite that of one, or both, of the conductive pads. In some embodiments, at least one conductive pad may be provided on a surface of the circuit board opposite that of at least one other conductive pad.

These embodiments discussed above provide considerable flexibility in creating the spark gaps. For example, all of the conductive structures shown in FIG. 2 may be formed on a common surface of the circuit board 40. In other embodiments, one or more, but not all, of the conductive pads 50-54 are provided on a different surface of the circuit from at least one other conductive pad, and thus, at least one of the traces from the pads to the spark gaps extend through the circuit board.

FIG. 6 shows an embodiment of a portion of the IMD 10 focusing on the header 18 mated to the can 29. The header 18 preferably is formed from plastic or other biocompatible material. Within the header 18 are included one or more connectors 80 to which leads 16 connect. The connectors 80 electrically connect to a conductive feedthrough component 150 that protrudes through an opening in the can 29 and into the header 18. The feedthrough component 150 includes a pair of conductive pins 152 and 154 to which wires (not shown) connect from the connector 80. Each conductor pin 152, 154 electrically couples to a corresponding pin 156, 158 on the opposite end of the feedthrough component 150. Conductive pin 152 couples to pin 156, while pin 154 couples to pin 158. Another pin 160 electrically connects to a conductive side surface of the feedthrough component 150. The side of component 150 is in electrical contact with the can 29. Conductive pins 156,158, and 160 mate to corresponding pads 50, 52, and 54, respectively, on the circuit board 40 by way of through-holes formed through the circuit board.

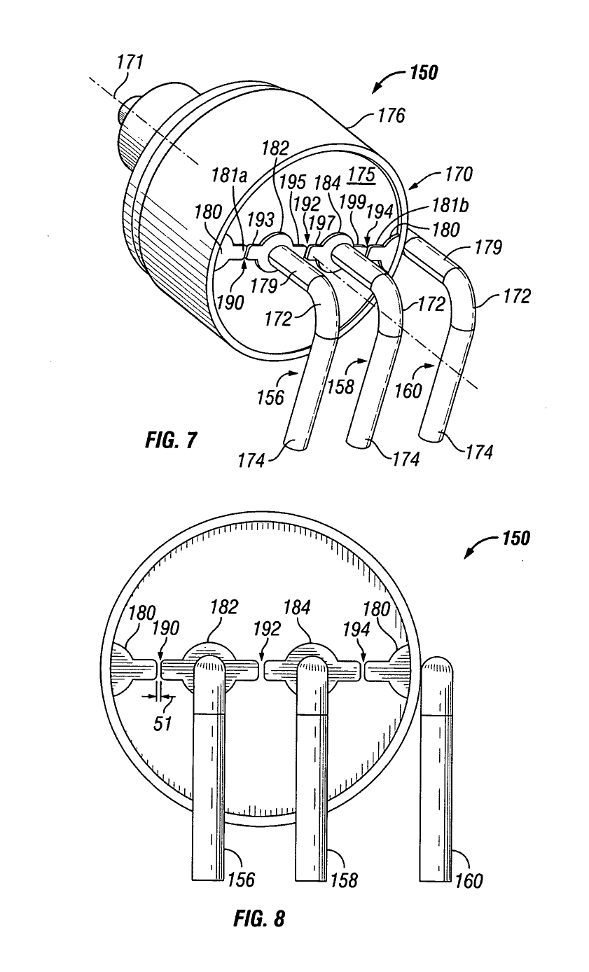

FIG. 7 shows an isolated perspective view of feedthrough component 150. In particular, the view of FIG. 7 shows an end portion of the feedthrough component 150 containing the conductive pins 156-160. Each conductive pin 156-160 includes sections 174 positioned orthogonal to a longitudinal axis 171 of the component 150. Each conductive pin also comprises a curved section 172 that transitions the orthogonal sections 174 to parallel sections 179 (parallel relative to longitudinal axis 171).

Each parallel section 179 electrically couples to a circuit board 175 provided at or near the end portion 170 of the feedthrough component 150. The circuit board 175 includes a plurality of conductive elements, such as elements 180, 182, and 184. Conductive elements 180-184 preferably are provided as conductive traces on circuit board 175. Conductive elements 182 and 184 comprise a conductive pad to which corresponding parallel linear sections 179 of pins 156 and 158 electrically couple. Pin 160 electrically couples to a conductive side surface 176 of the feedthrough component 150. Preferably, two separate conductive elements 180 electrically couple to the conductive side surface 176. Conductive elements 180 include extension portions 181a and 181b that preferably extend from the side surface 176 toward the conductive elements 182 and 184 as shown. Conductive element 182 includes a pair of extension portions 193 and 195, while conductive element 184 includes a pair of extension portions 197 and 199. The spacing between extension portions 181a and 193 define a spark gap 190. Similarly, the spacing between extension portions 195 and 197 and between extension portions 199 and 181b define spark gaps 192 and 194, respectively.

The spark gaps 190, 192, and 194 in FIG. 7 serve the same or similar purpose as the spark gaps implemented on the circuit board 40 within the can (FIG. 2) in that ESD imposed on one electrode/lead will arc across the spark gap rather than damaging the IMD's electronics. The difference is the location of the spark gaps. In FIG. 2, the spark gaps are formed on a surface of the circuit board 40 contained within the can 29, whereas in FIG. 7, the spark gaps are formed on a circuit board, or other suitable structure, in or coupled to the feedthrough component 150.

FIG. 8 shows a plan view of the end portion 170 of the feedthrough component 150. The size and shape of the spark gaps 190, 192, and 194 can be the same as or similar to the spark gaps 56, 58, and 60 of FIG. 2. That is, the size of each spark gap, as denoted by Si in FIG. 8, may be approximately 0.002 inches to 0.004 inches, and, in some embodiments, preferably approximately 0.003 inches.

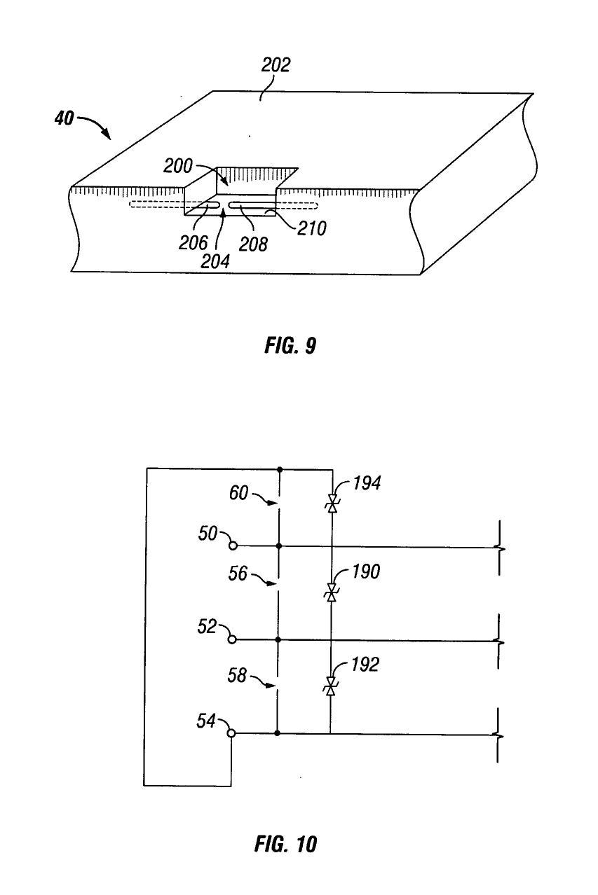

In accordance with the preferred embodiments, the IMD 10 includes at least one spark gap between at least two conductors associated with the electrodes/leads. Each spark gap preferably is exposed to the gas contained within the can 29 to thereby facilitate the electrical arc in the presence of an excessive level of ESD. In some embodiments, a spark gap is provided on a surface of circuit board, be it a circuit board contained within the can or a circuit board within or mated to the feedthrough component 150. In other embodiments, a spark gap could be implemented within a cavity formed in a circuit board wherein the cavity is preferably exposed to the gas. FIG. 9, for example, shows a cross sectional perspective view of the circuit board 40 in which a cavity 200 is formed in an exposed surface 202. In the cavity 200 an internal conductive layer 210 of the circuit board 40 is exposed to the gas contained within the can 20. In the example of FIG. 9, a spark gap 204 is formed between a pair of conductive trace ends 206 and 208. As such, the spark gap 204 is exposed to the gas within the can and can thus permit an electrical arc to occur as explained above.

In accordance with another embodiment of the invention, a diode is coupled to the conductive pads and across (e.g., in parallel with) a spark gap. In the embodiment shown in FIG. 10, for example, a diode is provided across each of the spark gaps (e.g., one diode per spark gap). Diode 190 is provided across spark gap 56, while diodes 192 and 194 are provided across spark gaps 58 and 60, respectively.

Preferably, each diode comprises a surge suppression diode implemented in the form of back-to-back zener diodes. Such a diode configuration is bidirectional meaning that the diode device will turn on and conduct current when the voltage exceeds a threshold (which can be any desired threshold and in some embodiments is 25 volts) regardless of the polarity. For example, diode 190 will turn on if the voltage on conductive pad 50 with respect to conductive pad 52 exceeds, for example, positive or negative 25 volts.

Without limiting the scope of this disclosure and the claims that follow, surge suppression diodes work generally well at lower voltages, while the spark gaps generally work well at higher voltage, higher current situations. The combination of diodes and spark gaps provides better performance compared to the use of diodes alone or the use of spark gaps alone.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. An implantable medical device, comprising:
an enclosure containing a gas; and
a plurality of conductors electrically coupled to tissue;
wherein at least two of said conductors define a spark gap formed therebetween and exposed to said gas.

2. The implantable medical device of claim 1 wherein said gas comprises an inert gas.

3. The implantable medical device of claim 1 wherein said spark gap has a length of approximately from 0.002 inches to 0.004 inches.

4. The implantable medical device of claim 1 wherein said spark gap has a length of approximately 0.003 inches.

5. The implantable medical device of claim 1 wherein said spark gap is formed on an exposed surface of a circuit board.

6. The implantable medical device of claim 1 further comprising a feedthrough component associated with said enclosure, wherein said plurality of conductors and said spark gap are located on said feedthrough component.

7. The implantable medical device of claim 6 wherein said feedthrough component comprises a circuit board on which said spark gap is defined.

8. The implantable medical device of claim 1 wherein at least two of said plurality of conductors comprises an end, said spark gap is formed between said ends, and each end comprises a shape that is selected from curved, square, pointed, and combinations thereof.

9. The implantable medical device of claim 1 wherein said plurality of conductors comprises at least three conductors and wherein each of at least two pairs of conductors defines a spark gap.

10. The implantable medical device of claim 1 wherein said spark gap is defined between a conductor electrically coupled to a lead and another conductor electrically coupled to the enclosure.

11. The implantable medical device of claim 1 wherein said spark gap is defined between two conductors that each are electrically coupled to a lead.

12. The implantable medical device of claim 1 further comprising a diode coupled to at least two of said plurality of conductors and across said spark gap.

13. The implantable medical device of claim 1, wherein said enclosure comprises a can.

14. An implantable medical device, comprising:
a can;
a circuit board contained within the can;
control logic provided on said circuit board;
a plurality of connection points, each connection point adapted to couple to one of a lead and the can;
a plurality of conductive elements, each conductive element electrically coupled to a connection point; and
a gap formed between two conductive elements on an exposed surface of said circuit board, said gap configured to permit an electrostatic discharge to arc from one of the two conductive elements to the other of the two conductive elements when a voltage on one of the conductive elements exceeds a safety threshold for the medical device.

15. The implantable medical device of claim 14 wherein said conductive elements comprise traces on said circuit board.

16. The implantable medical device of claim 15 wherein at least one of said conductive elements comprises an end that has a shape selected from a group consisting of round, square, pointed, and combinations thereof.

17. The implantable medical device of claim 14 further comprising an inert gas contained within the can and wherein said gap is exposed to said inert gas.

18. The implantable medical device of claim 14 wherein said gap has a length of approximately from 0.002 inches to 0.004 inches.

19. The implantable medical device of claim 14 further comprising a diode coupled to two of said conductive elements and coupled across said gap.

20. The implantable medical device of claim 19 further comprising a plurality of diodes, wherein each said diode is coupled to two of said conductive elements.

21. A circuit board adapted to be housed within an enclosure of an implantable medical device, comprising:
control logic provided on said circuit board;
a plurality of connection points, each connection point adapted to couple to one of a lead and an enclosure; and
at least two conductive elements each coupled to one of said plurality of connection points, and defining a spark gap on an exposed surface of said circuit board, said spark gap being configured to permit an electrostatic discharge to arc from a first conductive element to a second conductive element when a voltage on one of the conductive elements exceeds a safety threshold.

22. The circuit board of claim 21 wherein said spark gap has a length of approximately from 0.002 inches to 0.004 inches.

23. The circuit board of claim 21 further comprising a diode coupled to said at least two conductive elements and coupled across said spark gap.

24. The circuit board of claim 21 wherein said conductive elements comprise traces on said circuit board.

25. The circuit board of claim 21 wherein at least one of said conductive elements comprises an end that has a shape selected from a group consisting of curved, square, and pointed, and combinations thereof.
